(19) United States
(12) Patent Application Publication
(10) Pub. No.: US 2024/0068053 A1
(43) Pub. Date: Feb. 29, 2024

(54) SPECIFIC DETECTION OF NUCLEIC ACID SEQUENCES USING ACTIVATE CLEAVE & COUNT (ACC) TECHNOLOGY

(71) Applicant: The Board of Trustees of the University of Illinois, IL (US)

(72) Inventors: Brian T. CUNNINGHAM, Champaign, IL (US); Anurup GANGULI, Urbana, IL (US); Taylor D. CANADY, Champaign, IL (US); Nantao LI, Champaign, IL (US); Ying XIONG, Changsha (CN); Shreya GHOSH, Champaign, IL (US); Yanyu XIONG, Champaign, IL (US); Lucas D. AKIN, Champaign, IL (US)

(21) Appl. No.: 18/260,029

(22) PCT Filed: Dec. 31, 2021
(86) PCT No.: PCT/US2021/065804

§ 371 Date: Jun. 29, 2023

Related U.S. Application Data
(60) Provisional application No. 63/132,836, filed on Dec. 31, 2020.

(51) Int. Cl. C12Q 1/70; C12Q 1/6818

(52) U.S. Cl. CPC C12Q 1/701; C12Q 1/6818

(57) ABSTRACT

The current disclosure provides a simple single-step room temperature Activate Cleave and Count (ACC) assay coupled to Photonic Resonator Absorption Microscopy (PRAM) in an amplification-free approach. The assay, and associated system and method disclosed herein allow for detection of viral and bacterial pathogens as well disease such as cancer at the point of care.

### BACKGROUND

Since the SARS-CoV-2 (COVID-19) virus jumped from an animal reservoir to humans in December 2019, the virus has rapidly spread across the world, bringing death, illness, disruption to daily life, and economic losses to businesses and individuals. A key challenge of the health system across every country has been the ability to diagnose the disease rapidly and accurately, with contributing factors that include a limited number of available test kits, a limited number of certified testing facilities, combined with the length of time required to obtain a result and provide information to the patient. The challenges associated with rapid diagnostic testing contribute to uncertainly surrounding which individuals should be quarantined, sparse epidemiological information, and inability to quickly trace pathogen transmission within/across communities. The challenges underlying COVID-19 diagnosis are already well known from encounters with previous newly emerging epidemics and pandemics and are also representative of the challenges inherent in diagnosing mosquito-borne diseases (Zika, Dengue, Chikungunya, Malaria), HIV, and others. Already, the ability to perform pervasive testing has shown clear benefits to countries that implement it, such as South Korea, to provide accurate information regarding whom to quarantine, which in turn results in more timely control of disease propagation. Even after the current initial first wave of COVID-19 infections, continuing surveillance is expected to continue, and likely become more a routine aspect of travel, employment, and the many situations that require close person-to-person interactions.

However, available technologies remain expensive (in terms of instrument capital equipment and reagents), technically challenging, and labor intensive. As such, there is an urgent need for low-cost portable platforms that can provide fast, accurate, and multiplexed diagnosis of infectious disease at the point of care. Polymerase Chain Reaction (PCR) [1][2][3] and related approaches suffer from high false negative rates due to a combination of a low amount of starting material (one genome copy per viral particle), instability of the RNA extraction process, inhibiting substances in the test sample, and quality control failure of the many reagents [4][5]. In addition, enzymatic DNA/RNA amplification techniques suffer from false positives when working from minimally processed samples at the point-of-care due to primer dimerization and disruption of ideal buffer conditions [6].

Further, detection of diseases suffers from similar limitations. For example, cancer diagnosis requires expensive, complex, time consuming tests to accurately detect the presence of cancer. This results in an unnecessary physical and emotional burden on the patient and contributes to rising health care costs.

Thus, there is a need in the art to provide low-cost portable platforms for detection of infectious diseases and pathologies such as cancer that are reliable, rapid, and inexpensive.

### SUMMARY

In one aspect, example embodiments provide a system for detecting nucleic acids in a sample. The system comprises a source substrate with streptavidin linked nanoparticles bound to the surface of the source substrate by nucleotide tethers; an assay medium comprising a guide polynucleotide sequence and a Cas enzyme, wherein the guide polynucleotide sequence and the Cas enzyme are capable of forming a CRISPR/Cas complex when exposed to a sample containing a target nucleotide sequence; a biotinylated biosensor; and an imaging platform. The guide polynucleotide sequence binds the target nucleotide sequence and Cas enzyme thereby forming the CRISPR/Cas complex and the Cas enzyme is configured to cleave the nucleotide tethers thereby releasing the streptavidin linked nanoparticles which are then able to bind the biotinylated biosensor followed by use of an imaging platform that is configured to quantify the number of streptavidin linked nanoparticles bound to the biotinylated biosensor.

In a further aspect, example embodiments provide a biologic assay comprising a source substrate; a biotinylated biosensor; assay medium comprising a guide polynucleotide sequence and a Cas enzyme, a population of streptavidin linked nanoparticles; and a plurality of nucleotide tethers; wherein the streptavidin-linked nanoparticles are bound to the biosensor using the plurality of nucleotide tethers, and wherein the nucleotide tethers are comprised of a nucleic acid sequence.

In yet a further aspect, example embodiments provide a method for detecting nucleic acids in a sample, wherein streptavidin is bound to a nanoparticle to create a streptavidin containing nanoparticle. The streptavidin containing nanoparticles are bound to the surface of a source substrate using nucleotide tethers, thereby creating an assay surface. A biotinylated biosensor is produced by coating a biosensor with biotin. An activated Cas enzyme is generated by adding a test sample to an assay medium, wherein the assay medium comprises a guide polynucleotide sequence and a Cas enzyme and wherein the guide polynucleotide sequence and the Cas enzyme are capable of forming an activated CRISPR/Cas complex when exposed to the test sample containing a target nucleotide sequence. Streptavidin containing nanoparticles, cleaved upon incubation of the activated Cas enzyme and assay surface; are then captured and incubated with the biotinylated biosensor; and the number of streptavidin containing nanoparticles that bind the biotinylated biosensor quantified using an imaging platform.

In one aspect, example embodiments provide a system for detecting nucleic acids in a sample, comprising streptavidin linked nanoparticles bound to free floating microparticles by nucleotide tethers. The system also comprises an assay medium comprising a guide polynucleotide sequence and a Cas enzyme, wherein the guide polynucleotide sequence and the Cas enzyme are capable of forming a CRISPR/Cas complex when exposed to a sample containing a target nucleotide sequence, a biotinylated biosensor, and an imaging platform. The In the system, a guide polynucleotide sequence binds the target nucleotide sequence and Cas enzyme thereby forming the CRISPR/Cas complex wherein the Cas enzyme is configured to cleave the nucleotide tethers thereby releasing the streptavidin linked nanoparticles. The streptavidin linked nanoparticles then bind the biotinylated biosensor, and the imaging platform configured to quantify the number of streptavidin linked nanoparticles bound to the biotinylated biosensor.

In a further aspect is a biologic assay comprising streptavidin linked nanoparticles, free floating microparticles, a biotinylated biosensor, an assay medium comprising a guide polynucleotide sequence and a Cas enzyme, a population of streptavidin linked nanoparticles, and a plurality of nucleotide tethers. The streptavidin-linked nanoparticles are bound to the free floating microparticles using the plurality of nucleotide tethers, and the nucleotide tethers are comprised of a nucleic acid sequence.

In yet a further aspect is a method for detecting nucleic acids in a sample comprising binding streptavidin to a nanoparticle to create a streptavidin containing nanoparticle and tethering the streptavidin containing nanoparticles to free floating microparticles using nucleotide tethers. A biotinylated biosensor is created by coating a biosensor with biotin. An activated Cas enzyme is generated by adding a test sample to an assay medium, wherein the assay medium comprises a guide polynucleotide sequence and a Cas enzyme. The guide polynucleotide sequence and the Cas enzyme are capable of forming an activated CRISPR/Cas complex when exposed to the test sample containing a target nucleotide sequence and streptavidin containing nanoparticles cleaved upon incubation of the activated Cas enzyme and free floating microparticles are captured and incubated with the cleaved streptavidin containing nanoparticles with the biotinylated biosensor. The streptavidin containing nanoparticles that bind the biotinylated biosensor using an imaging platform are then quantified.

In one aspect, example embodiments provide a system for detecting nucleic acids in a sample. The system comprises a biosensor with nanoparticles bound to the surface of the biosensor by nucleotide tethers; an assay medium comprising a guide polynucleotide sequence and a Cas enzyme, wherein the guide polynucleotide sequence and the Cas enzyme are capable of forming a CRISPR/Cas complex when exposed to a sample containing a target nucleotide sequence; and an imaging platform. The guide polynucleotide sequence binds the target nucleotide sequence and Cas enzyme thereby forming the CRISPR/Cas complex. The Cas enzyme is configured to cleave the nucleotide tethers thereby releasing nanoparticles. The imaging platform is configured to quantify the number of nanoparticles tethered to the biosensor prior to and after addition of the sample.

In a further aspect, example embodiments provide a biologic assay comprising a biosensor; assay medium comprising a guide polynucleotide sequence and a Cas enzyme; a population of nanoparticles; and a plurality of nucleotide tethers. The nanoparticles are bound to the surface of the biosensor using the plurality of nucleotide tethers, and the nucleotide tethers are comprised of a nucleic acid sequence.

In yet another aspect, example embodiments provide a method for detecting nucleic acids in a sample. Detection is achieved by tethering nanoparticles to the surface of a biosensor using nucleotide tethers, thereby creating an assay surface. An assay medium is then added to the assay surface. The assay medium comprises a guide polynucleotide sequence and a Cas enzyme, wherein the guide polynucleotide sequence and the Cas enzyme are capable of forming a CRISPR/Cas complex when exposed to a sample containing a target nucleotide sequence. The method further comprises adding a biological sample that may contain the target nucleotide sequence to the assay, thereby forming a CRISPR/Cas complex and quantifying the number of nanoparticles tethered to the biosensor before and after addition of the sample using an imaging platform.

### BRIEF DESCRIPTION OF THE DRAWINGS

FIG. 1A is a schematic diagram of a portable PRAM detection instrument for illuminating a photonic crystal (PC) from below while gold nanoparticles (AuNPs) attach from above.

FIG. 1B is a peak intensity value (PIV) image of attached AuNPs.

FIG. 1C demonstrates a reduction in resonant reflection intensity of the PC due to one AuNP.

FIG. 2 illustrates the Activate Cleave and Count (ACC) assay concept. The activated CRISPR/Cas RNP complex cleaves DNA tether on PC surface resulting in the detachment of AuNPs and causing signal change.

FIG. 3A illustrates indiscriminate cleavage of reporter gene caused by RNP complex in the presence of 100 nM N gene.

FIG. 3B illustrates indiscriminate cleavage of reporter gene caused by RNP complex in the presence of 100 nM E gene.

FIG. 3C illustrates an increase in the fluorescence emission from FAAM after the addition of 100 nM of each of the target genes.

FIG. 3D illustrates fluorescence emission intensities as a function of the target N gene concentration.

FIG. 4A demonstrates cleavage of DNA tether and removal of nanoparticles after adding RNP complex targeting the N gene (100 nM) of the SARS-CoV-2 genome.

FIG. 4B demonstrates cleavage of DNA tether and removal of nanoparticles after adding RNP complex targeting the E gene (100 nM) of the SARS-CoV-2 genome.

FIG. 5A demonstrates the change in AuNP counts on PC before and after the addition of control sample (inactivated Cas12a/gRNA complex) for N gene and E gene respectively.

FIG. 5B is a comparative chart representing the relative change in AuNPs in the presence of RNP complexes containing N gene, Control (N gene), E gene and Control (E gene) respectively.

FIG. 6 is a schematic overview of AuNP Capture with Biotinylated PEG followed by activated cleavage and counting upon the release of streptavidin linked AuNP and subsequent binding to a biotinylated biosensor.

FIGS. 7A-7F are dose response curves from low concentration studies. Dose response curves of AuNPs bound versus target concentration are shown for 0.1 aM (approximately 300 copies of the target molecule in the test sample) (FIG. 7A), 1 aM (approximately 3,000 copies of the target molecule in the test sample) (FIG. 7B), 10 aM (approximately 30,000 copies of the target molecule in the test sample) (FIG. 7C), 100 aM (approximately 300,000 copies of the target molecule in the test sample) (FIG. 7D), and 1 fM (approximately 3,000,000 copies of the target molecule in the test sample) (FIG. 7E). Individual binding curves are graphed on a dose response curve of AuNPs bound versus target concentration (FIG. 7F).

FIG. 8. ACC dose response and target selectivity (Data Set 1 of 2). Images of PC surfaces with AuNPs captured following release by Cas12a after activation with (FIG. 8a) 1 zM, (FIG. 8b) 10 zM, (FIG. 8c) 100 zM or (FIG. 8d) 1 aM EGFR gene fragments. Left and right panels show AuNPs captured using EGFR^{WT} and EGFR^{L858R}, respectively. (FIG. 8e) Plot of AuNP counts from each image set (FIGS. 8a-8d).

FIG. 9. ACC dose response and target selectivity (Data Set 2 of 2). Images of PC surfaces with AuNPs captured following release by Cas12a after activation with (FIG. 9a) 1 zM, (FIG. 9b) 10 zM, (FIG. 9c) 100 zM or (FIG. 9d) 1 aM EGFR gene fragments. Left and right panels show AuNPs captured using EGFR^{WT} and EGFR^{L858R}, respectively. (FIG. 9e) Plot of AuNP counts from each image set (FIG. 9a-9d).

### DETAILED DESCRIPTION

It is to be understood that the particular aspects of the specification are described herein are not limited to specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements, or steps but not the exclusion of any other component, feature, element, or step or group of components, features, elements, or steps. Any of the terms “comprising”, “consisting essentially of”, and “consisting of” may be replaced with either of the other two terms, while retaining their ordinary meanings.

As used herein, the singular forms “a,” “an,” and “the” include plural referents unless the context clearly indicates otherwise.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein and in the drawings, ranges and amounts can be expressed as “about” a particular value or range. About also includes the exact amount. For example, “about 5%” means “about 5%” and also “₅%.” The term “about” can also refer to +10% of a given value or range of values. Therefore, about 5% also means 4.5%-5.5%, for example.

“Sample” as used herein refers to any type of sample, containing a nucleotide sequence and encompasses biological sample. “Biological sample” refers to a sample of body tissue, including but not limited to an organ punch or tissue biopsy, or fluid, including but not limited to blood, cerebrospinal fluid, plasma, or saliva from a warm-blooded animal such as a mammal, preferably a human, which is afflicted with, or has the potential to be afflicted with one or more diseases and/or disorders described herein. A biological sample can also refer to tissue or blood samples obtained from non-human mammals and other animals.

In view of the present disclosure, the methods and compositions described herein can be configured by the person of ordinary skill in the art to meet the desired need.

1. Overview

The current disclosure provides simple Activate Cleave & Count (ACC) assays coupled to an inexpensive portable instrument for detection of SARS-CoV-2 via targeting two independent and unique sections of its genome by using clustered regularly interspaced short palindromic repeats (CRISPR)-based nucleic acid detection coupled with Photonic Resonator Absorption Microscopy (PRAM) in an approach that does not use enzymatic amplification of the target nucleic acid sequence. The disclosed assays and detection instrument can also be adapted to detect the presence of a wide range of infectious agents other than SARS-CoV-2 as well as pathological diseases such as cancer. The PRAM instrument is described in U.S. patent application Ser. No. 16/170,111 while various aspects of photonic crystal (PC) biosensors are described in U.S. Pat. Nos. 7,479,404, 7,521,769, 7,531,786, 7,737,392, 7,742,662, and 7,968,836, all of which are incorporated herein by reference.

Microbial CRISPR and CRISPR-associated (CRISPR/Cas) adaptive immune systems contain programmable endonucleases that can be leveraged for CRISPR-based diagnostics [7][8]. The systems, assays, and methods described herein utilize the indiscriminate single stranded nucleic acid cleaving ability of these enzyme-guide RNA complexes (called RNP) after binding to its specific target (RNP activation), to generate a signal change. However, current platforms require a pre-amplification step using sequence-specific primers and a DNA polymerase for a measurable change to be detected on lateral flow test strips or fluorimeters from the CRISPR step. The current disclosure utilizes the PRAM biosensor imaging platform to perform digital counting of nanoparticles, including AuNPs bound to the photonic crystal (PC) nanostructured surface with a nucleic acid tether [9] or streptavidin-linked AuNPs bound to a biotinylated biosensor, to perform rapid detection of specific target nucleic acid sequences.

2. PRAM Working Principle

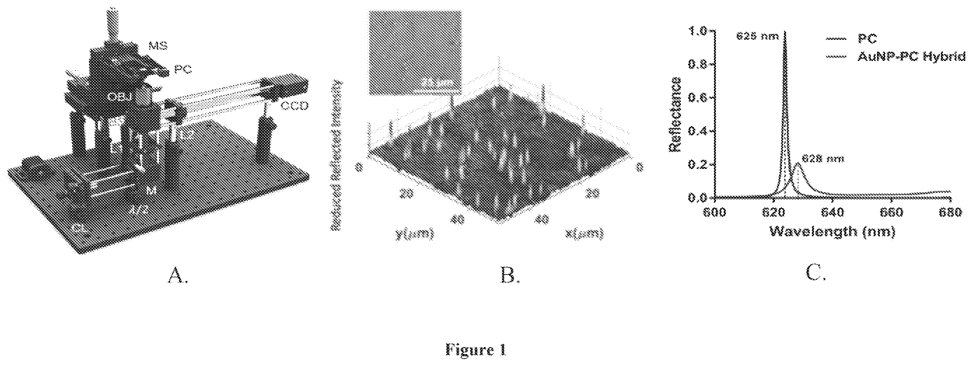

The portable version of the PRAM biosensing platform is illustrated in FIG. 1A. Port 1 is coupled to a fiber-coupled 617 nm LED light source (M617F2, Thorlabs), and a lens group (F810SMA-635, Thorlabs) is first utilized to collimate the output beam. A zero-order half-wave plate (WPH10M-633, Thorlabs) rotates the polarization of the collimated beam in order to excite the TM resonance mode of the PC cavity. A plano-convex lens (LA1509-A-ML, Thorlabs) then focuses the beam onto the back focal plane of an Olympus plan-fluorite objective 20×/0.5 numerical aperture (NA) objective, from which a collimated beam impinges onto the PC surface at normal incidence. A manual three-axis stage (PT3, Thorlabs) is used to secure the PC sample at the focal plane of the objective. The reflected light from the PC resonator is the collected by the same objective and redirected by a 50/50 non-polarizing beam-splitter (CCM1-BS013, Thorlabs). A doublet (AC254-200-A-ML, Thorlabs) projects the image plane onto a charge coupled device (CCD) camera (GS3-U3-51S5M-C, Point Grey), with a resolution of 177 nm/pixel. As shown in FIG. 1C, at a particular resonant wavelength and incident angle, complete interference occurs and no light is transmitted, resulting in nearly 100% reflection efficiency. The resonant reflectance magnitude is dramatically reduced (FIG. 1C) by the addition of absorbing AuNPs upon the PC surface, resulting in the ability to observe each AuNP by illuminating with light from an LED and making images of the reflected intensity (FIG. 1). By measuring the resonant peak intensity value (PIV) on a pixel-by-pixel basis across the PC using a microscopy approach called Photonic Resonator Absorption Microscopy (PRAM), PIV images of attached AuNPs may be gathered by illuminating the structure with collimated broadband light through the transparent substrate, while the front surface of the PC is immersed in aqueous media.

2. Assay Working Principle

The Activate Cleave and Count Assay (“Assay”) is an amplification-free biological assay, CRISPR-Cas based detection coupled to a PRAM biosensor imaging platform. In a first exemplary embodiment the platform performs digital counting of streptavidin linked gold nanoparticles (AuNP), that bind a biotinylated biosensor. In a second exemplary embodiment the platform performs digital counting of AuNPs released from a photonic crystal surface when the target nucleic acid sequence interacts with a guide polynucleotide sequence and a Cas enzyme to form an activated complex.

A first embodiment of the assay is a biotinylated nanoparticle capture assay wherein a PRAM instrument is used to detect the number of streptavidin linked nanoparticles that bind a biotinylated biosensor. The biotinylated nanoparticle capture assay is comprised of a source substrate, a population of nanoparticles linked to streptavidin with open pockets for biotin binding, a plurality of nucleotide tethers, assay medium comprising a guide polynucleotide sequence and a Cas enzyme, and a biotinylated biosensor. “Open pockets” as used herein, refers to one or more of the biotin binding sites on streptavidin that is available for biotin binding. More specifically, the source substrate contains a population of streptavidin linked nanoparticles wherein the nanoparticles are bound to the source substrate by way of the plurality of nucleotide tethers. The term “source substrate” refers to any biologically inert solid material selected from materials including glass (silicon oxide), plastic (polyester, polystyrene, acrylic), metal (gold, silver), or dielectric (silicon nitride or titanium oxide). The source substrate is a surface that can hold nanoparticles in close proximity to its surface with one or more ssDNA tethers. In one embodiment the source substrate is a PC biosensor.

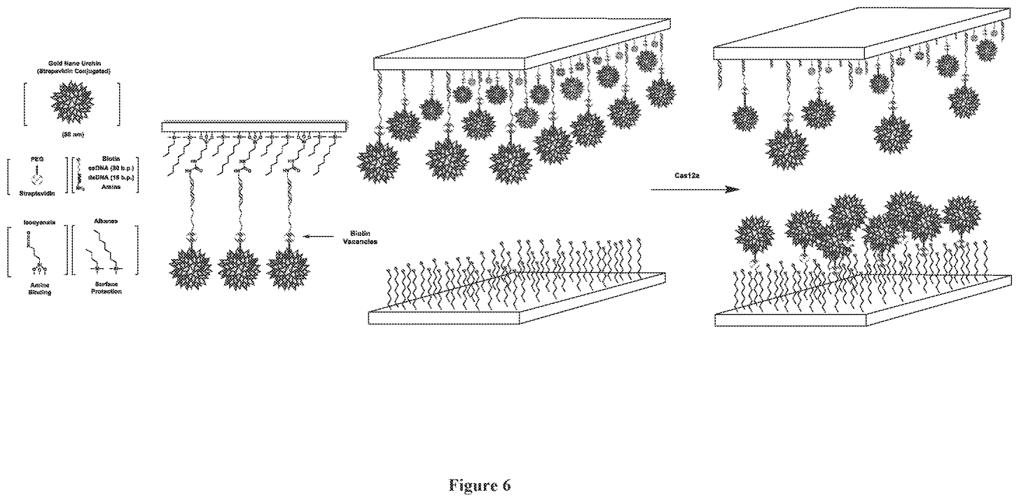

The nanoparticles can be comprised of a wide range of materials. In an exemplary embodiment the nanoparticles are gold nanoparticles (AuNP). In other embodiments the nanoparticle material is quantum dots, metal-based nanoparticles, magnetic nanoparticles, or nanoparticles comprised of dielectric materials such as SiO₂ or TiO₂. Magnetic-plasmonic nanoparticle tags can also be used thereby reducing the time required for the biosensor to bind the nanoparticle by applying an attractive magnetic field between the released nanoparticles and the biotinylated the biosensor. The streptavidin containing nanoparticles of the current disclosure are tethered to the source substrate using DNA nucleotide tethers comprised of a non-specific nucleotide sequence. Consistent with this, the tether can be almost any single stranded DNA sequence. A portion of tether may also be dsDNA, as shown in FIG. 6, thereby providing rigidity and to control the height displacement between the nanoparticle and the substrate. The nucleotide tethers can be homogenous or heterogenous in sequence and of a non-specific length. In some embodiments the nucleotide tethers are about 5 to 200 nucleotides in length. In some embodiments the tethers are about 5-50, about 51-100, about 101-150, or about 151-200 nucleotides in length. In yet further embodiments the nucleotide tethers are about 5-25, about 26-50, about 51-75, about 76-100, about 101-125, about 126-150, about 151-175, or about 176-200 nucleotides in length. Both ends of the tether can be prepared with chemical functional groups that facilitate formation of covalent chemical bonds or biotin-streptavidin association with the source substrate and the nanoparticle on opposing ends of the tether.

In an exemplary embodiment streptavidin is linked or attached to the nanoparticle, preferably an AuNP, using PEGylation or other methods known in the art for covalent or non-covalent attachment of streptavidin. A second biotin binding site on the streptavidin is utilized to bind the nucleotide tether, thereby creating a nanoparticle-nucleotide tether-source substrate linkage, as presented in FIG. 6. In a preferred embodiment the tether, preferably ssDNA, tethers the streptavidin linked AuNP to the source substrate via isocyanates. In alternate embodiments the ssDNA is tethered to the streptavidin linked nanoparticle, such as a AuNP, via alkyl halides, sulfonates, aldehydes, carboxylic acids, or epoxides.

The biotinylated nanoparticle capture assay disclosed herein allows for the detection of the presence of one or more target RNA or DNA molecules, whose sequence is a biomarker for disease, the presence of a viral pathogen, or the presence of a bacterial pathogen. Consistent with this, in an exemplary embodiment, a sample and/or biological sample, suspected of having a target nucleotide sequence and thereby being complementary to the guide polynucleotide sequence, and capable of forming an activated CRISPR/Cas complex, is incubated with the guide polynucleotide sequence and a Cas enzyme. The presence of the target molecule in the sample results in activated Cas, with the concentration of activated Cas directly proportional to the concentration of the target molecule. The Cas containing sample may contain both activated and non-activated Cas. Appropriate negative and positive controls can also be included in the reaction.

“Cas enzyme” as used herein, can include any Cas enzyme capable of forming a Cas/CRISPR complex. One of skill in the art will understand that Cas enzymes are classified into Class I and Class II. In a preferred embodiment the Cas Enzyme is a Class II enzyme, more specifically Cas9, Cas12a, Cas12b, or Cas13a. However, alternate Class II Cas enzymes can also be used as part of the assay, including but not limited to Csn2, Cas4, Cas12c, Cas12d, Cas12e, Cas12f, Cas12g, Cas12h, Cas12i, Cas12k, C2c4, C2c8, C2c9, Cas13b, Cas13c, or Cas13d. In preferred embodiments Cas9 is used to detect messenger RNA, Cas12 is used to detect double stranded DNA, and Cas13 is used to detect microRNA.

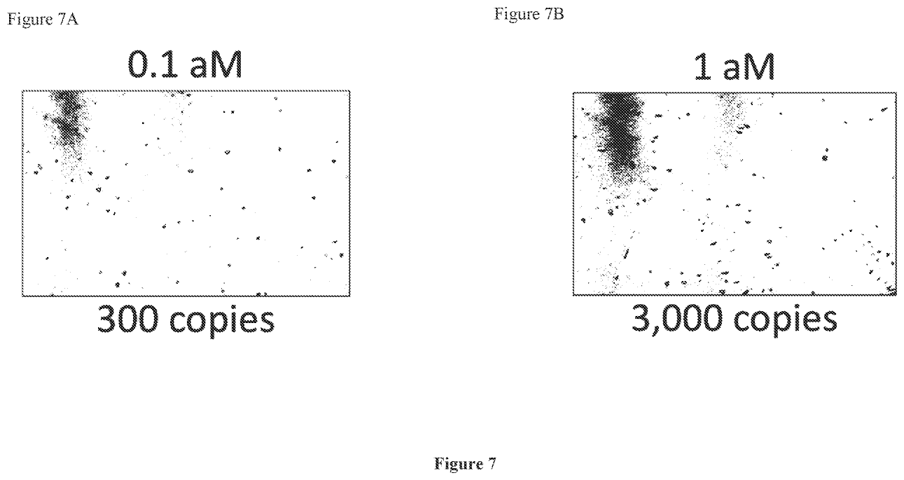

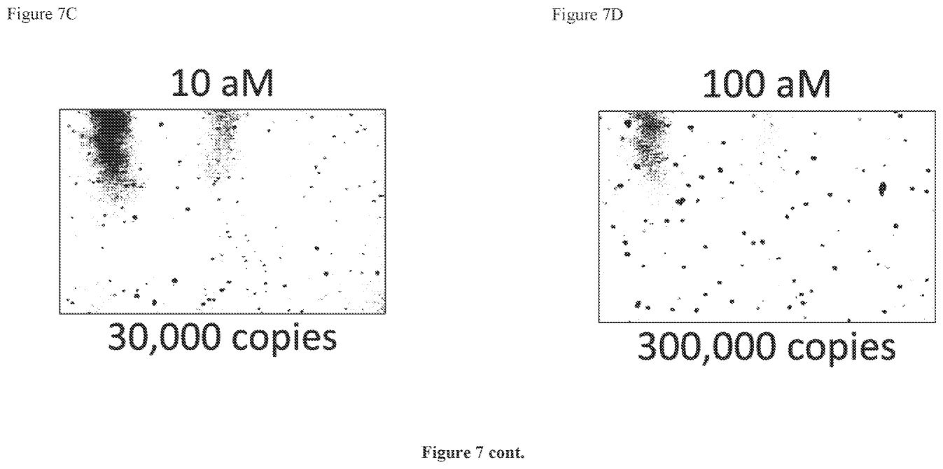

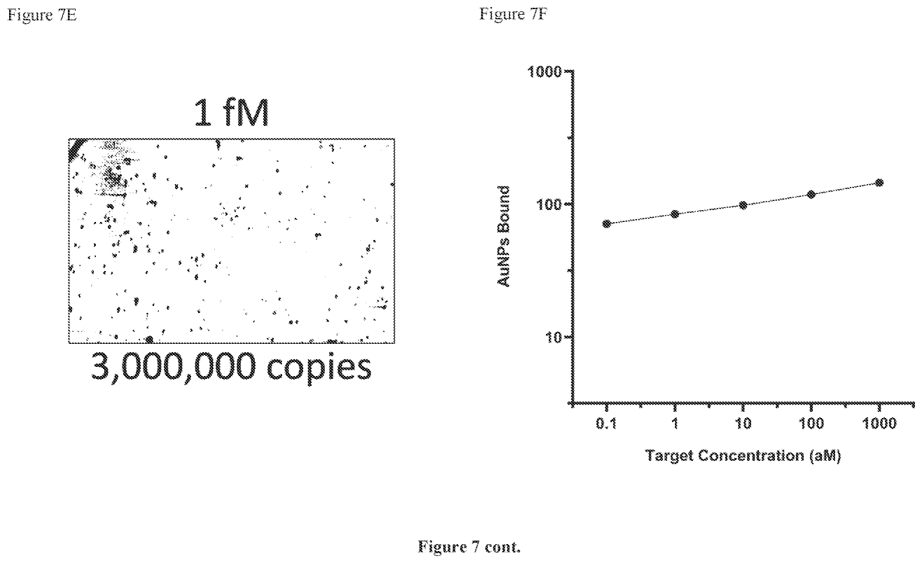

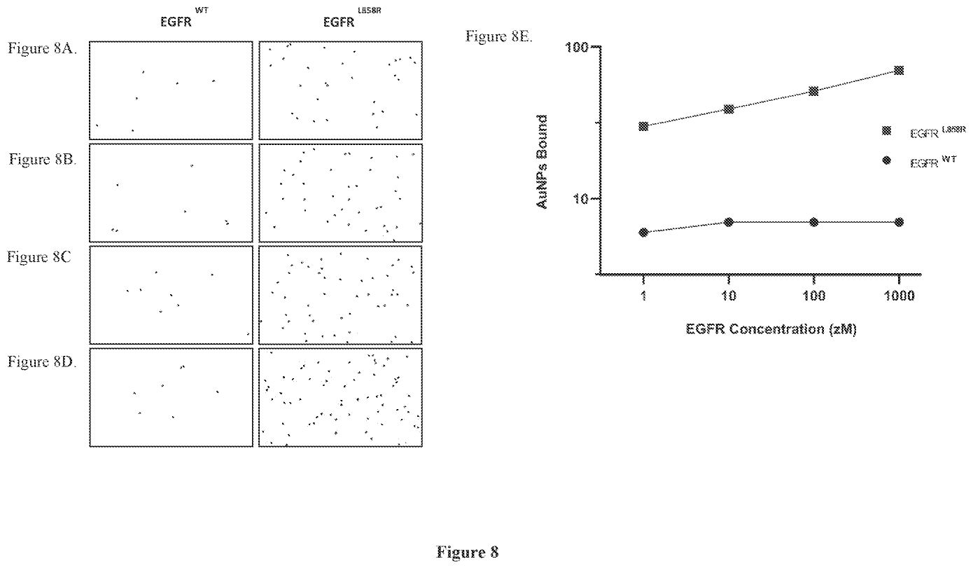

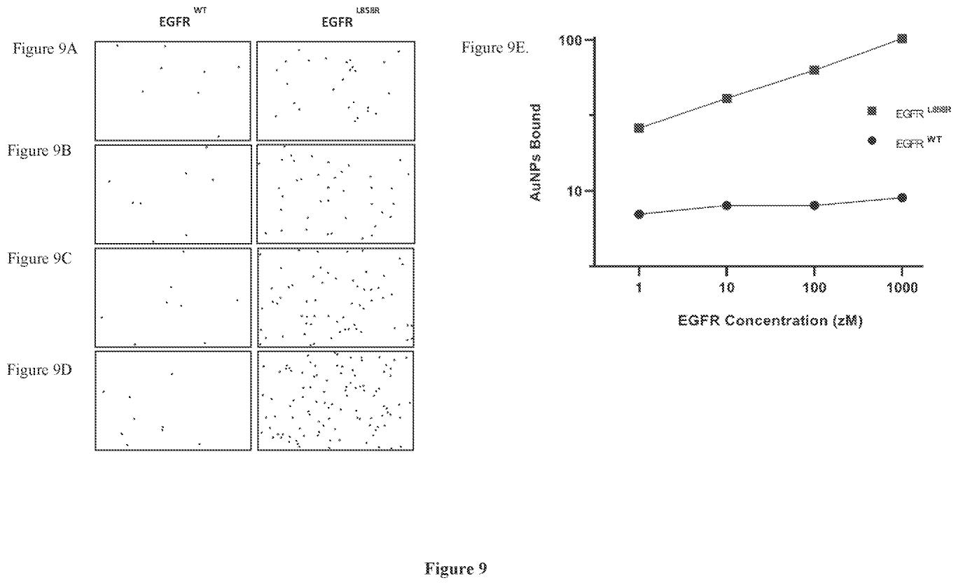

In the biotinylated nanoparticle capture assay the activated Cas sample is incubated with the source substrate containing the tethered streptavidin linked AuNPs. Activated Cas cleaves the ssDNA tether thereby releasing the streptavidin linked AuNPs into the assay medium. The assay medium containing the streptavidin linked AuNPs is incubated with the biotinylated biosensor, allowing for streptavidin-biotin binding via the open pockets and subsequent quantification of streptavidin-biotin binding via the PRAM instrument. The quantitative change in bound particles is indicative of the presence or absence of a disease, viral pathogen, or bacterial pathogen in the sample or biological sample. The detection limit of the biotinylated nanoparticle capture assay is 1 zM for the target ctDNA sequence (FIGS. 7-9), representing approximately 3 copies of the target DNA sequence in the test sample. Three copies of gene target suspended in 150 μL of solution equals 3.33×10⁻²⁰ M (33 zM). Because clinical plasma samples are commonly 5 mL in volume, three copies of gene target in a volume of 5 mL equals 1.00×10⁻²¹ (1 zM). Herein is reported a limit of detection of 1 zM as plasma DNA extraction kits elute 100 μL of purified DNA from up to 5 mL of human plasma. Hence, the reported limit of detection of 1 zM corresponds to the number of target gene copies present in 5 mL of plasma and by virtue assumes that all DNA present in the sample is isolated in a 100 μL elution volume, followed by final dilution to 150 μL for the subsequent cleavage step.

Preferably, the biosensor is a photonic crystal. The biosensor can also be a whispering gallery mode biosensor that is a ring resonator, microtoroid, or microsphere. In further embodiments the biosensor is a waveguide structure through which light travels laterally, an acoustic biosensor, a photoacoustic biosensor, or a surface plasmon resonant biosensor. In yet further embodiments, the AuNPs released from the source substrate are subsequently captured on a surface that is measured by other forms of microscopy to count the nanoparticles that are captured, such as electron microscopy, dark field microscopy, or reflection interference microscopy. If the nanoparticles are photon emitters, such as quantum dots or phosphorescent nanoparticles, the microscopy system may be a fluorescence microscope or total internal reflectance fluorescence microscope. FIG. 6 provides an overview of the AuNP Capture assay.

The streptavidin-linked nanoparticles can also be tethered to micrometer-scale particles that are free floating in a solution with activated Cas. The term “microparticles” as used herein refers to microparticles that are polymer beads, magnetic beads, or glass beads (silicon oxide) ranging in size from about 2-75 micrometers in diameter, about 2-70 micrometers in diameter, about 2-65 micrometers in diameter, about 2-60 micrometers in diameter, about 2-55 micrometers in diameter, about 2-50 micrometers in diameter, about 2-45 micrometers in diameter, about 2-40 micrometers in diameter, about 2-35 micrometers in diameter, about 2-30 micrometers in diameter, about 2-25 micrometers in diameter, about 2-20 micrometers in diameter, about 2-15 micrometers in diameter, about 2-10 micrometers in diameter, about 5-75 micrometers in diameter, about 5-70 micrometers in diameter, about 5-65 micrometers in diameter, about 5-60 micrometers in diameter, about 5-55 micrometers in diameter, about 5-50 micrometers in diameter, about 5-45 micrometers in diameter, about 5-40 micrometers in diameter, about 5-35 micrometers in diameter, about 5-30 micrometers in diameter, about 5-25 micrometers in diameter, about 5-20 micrometers in diameter, about 5-15 micrometers in diameter, or about 5-20 micrometers in diameter. Free floating micrometer-scale particles allows for diffusion of the microparticles and Cas in free solution, thereby allowing Cas to encounter more ssDNA tethers, allowing for cleavage of the ssDNA in a shorter time. In this embodiment the use of free floating microparticles requires an additional step after ssDNA cleaving wherein the microparticles are separated from the solution using centrifugation or magnets thereby segregating the released streptavidin-linked nanoparticles in the supernatant. The supernatant containing the streptavidin-linked nanoparticles is then incubated with the biotinylated biosensor, allowing for streptavidin-biotin binding via the open pockets and subsequent quantification of streptavidin-biotin binding via the PRAM instrument. The quantitative change in bound particles is indicative of the presence or absence of a disease, viral pathogen, or bacterial pathogen in the sample or biological sample.

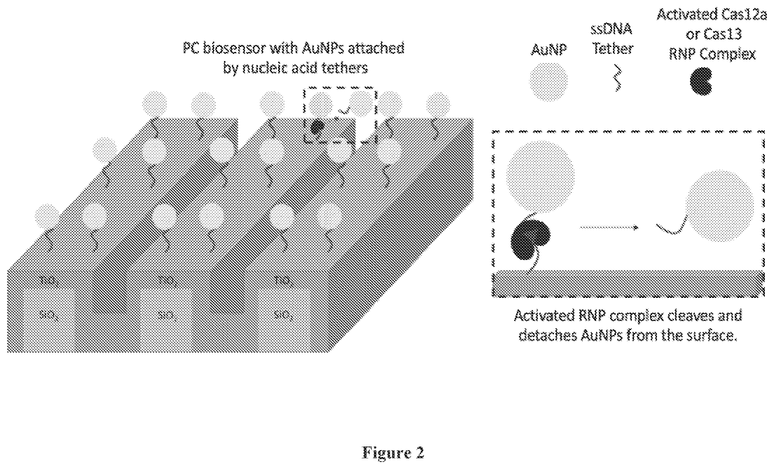

In a second embodiment, the assay disclosed herein operates on the principle of indiscriminate single stranded nucleic acid cleaving ability of the CRISPR/Cas enzyme-guide RNA complexes (called RNP) after binding to its specific target (RNP activation), to generate a signal change. In this embodiment, AuNPs are attached to the surface of a photonic crystal (PC) via DNA tethers. Upon binding to the specific SARS-CoV-2 RNA, the activated RNP complex non-specifically and repeatedly cleave the DNA tethers, thus releasing gold nanoparticles from the PC surface. The PRAM instrument then detects and counts each surface-released gold nanoparticles, providing an immediate readout of the presence of SARS-CoV-2 RNA in the test sample as shown in FIG. 2.

The second embodiment of the biological assay is comprised of a biosensor; assay medium comprising a guide polynucleotide sequence and a Cas enzyme, a population of nanoparticles; and a plurality of nucleotide tethers. The biosensor contains nanoparticles bound to the surface using the plurality of nucleotide tethers. The nanoparticles can be comprised of a wide range of materials, in one embodiment the nanoparticles are gold. In other embodiments the nanoparticle material is quantum dots, metal-based nanoparticles, magnetic nanoparticles, nanoparticles comprised of dielectric materials such as SiO₂ or TiO₂, or magnetic-plasmonic nanoparticle. The tether can be any RNA/DNA sequence.

The nanoparticles are tethered to the surface of the biosensor using nucleotide tethers comprised of a non-specific nucleotide sequence. In one embodiment the source substrate is a PC biosensor. The nucleotide tethers can be homogenous or heterogenous in sequence and of a non-specific length. In some embodiments the nucleotide tethers are about 5 to 200 nucleotides in length. In some embodiments the tethers are about 5-50, about 51-100, about 101-150, or about 151-200 nucleotides in length. In yet further embodiments the nucleotide tethers are about 5-25, about 26-50, about 51-75, about 76-100, about 101-125, about 126-150, about 151-175, or about 176-200 nucleotides in length.

The biosensor can be a photonic crystal. The biosensor can also be a whispering gallery mode biosensor that is a ring resonator, microtoroid, or microsphere. In further embodiments the biosensor is a waveguide structure through which light travels laterally, an acoustic biosensor, or a photoacoustic biosensor.

As noted previously, “Cas enzyme” as used herein, can include any Cas enzyme capable of forming a Cas/CRISPR complex. In a preferred embodiment the Cas Enzyme is a Class II enzyme, more specifically Cas9, Cas12a, Cas12b, or Cas13a, however, alternate Class II Cas enzymes can also be used as part of the assay, including but not limited to Csn2, Cas4, Cas12c, Cas12d, Cas12e, Cas12f, Cas12g, Cas12h, Cas12i, Cas12k, C2c4, C2c8, C2c9, Cas13b, Cas13c, or Cas13d. In preferred embodiments Cas9 is used to detect messenger RNA, Cas12 is used to detect double stranded DNA, and Cas13 is used to detect microRNA.

The second embodiment of the assay disclosed herein allows for the detection of the presence of target RNA or DNA molecule, whose sequence is a biomarker for disease, the presence of a viral pathogen, or the presence of a bacterial pathogen. Consistent with this, a sample and/or biological sample, suspected of having a target nucleotide sequence and thereby being complementary to the guide polynucleotide sequence and capable of forming an activated CRISPR/Cas complex is added to the assay. The activated complex then cleaves the nucleotide tether and the change in bound nanoparticle quantity determined using a PRAM instrument. The quantitative change in bound particles is indicative of the presence or absence of a disease, viral pathogen, or bacterial pathogen in the sample or biological sample. More specifically, the quantitative difference is calculated as the difference between the nanoparticles tethered to surface of the biosensor prior to and after the addition of the sample. The reduction in the number of tethered nanoparticles is indicative of the presence of a RNA or DNA molecule whose sequence is a biomarker for disease, a viral pathogen, or a bacterial pathogen. Further, the nucleotide sequence of interest can be indicative of the presence of a disease. SARS-CoV-2 and cancer are an exemplary viral pathogen and disease that can be detected using the system, assay, and/or method disclosed herein. Following quantification of the tethered nanoparticles the nanoparticles can be removed from the biosensor surface allowing for reuse of the biosensor. The nanoparticles can be removed from the biosensor surface by replacing the assay buffer or by agitation of the assay buffer without replacement of the assay buffer. The nanoparticle, if magnetic, can also be removed from the biosensor surface by application of a magnetic field. The assays described herein, can also be part of a system for detecting nucleic acids in a sample. The systems of the current disclosure are comprised of one or more of a source substrate, a biosensor with nanoparticles bound to the surface of the biosensor by nucleotide tethers or a biotinylated biosensor, an assay medium comprising a guide polynucleotide sequence and a Cas enzyme configured to cleave the nucleotide tethers, wherein the guide polynucleotide sequence and the Cas enzyme are capable of forming a CRISPR/Cas complex when a sample containing a target nucleotide sequence is added to the assay; and a PRAM imaging platform configured to quantify the number of nanoparticles tethered to the biosensor prior to and after addition of the sample.

The current disclosure also provides methods for using assays in detection of nucleic acid sequences of interest in a sample, such as nucleic acids sequences associated with infectious agents, pathogens, or disease. In a first exemplary embodiment is a method for detecting nucleic acids in a sample. Streptavidin is linked to the nanoparticle using PEGylation or other techniques for attachment, commonly understood in the art. The streptavidin containing nanoparticles are tethered to the surface of a source substrate using nucleotide tethers, thereby creating an assay surface and a biosensor coated with biotin. An assay medium is added to the assay surface, wherein the assay medium comprises a guide polynucleotide sequence and a Cas enzyme, wherein the guide polynucleotide sequence and the Cas enzyme are capable of forming an activated CRISPR/Cas complex when exposed to a sample containing a target nucleotide sequence. A biological sample that may contain the target nucleotide sequence to the assay is added, thereby forming an activated CRISPR/Cas complex that releases the streptavidin containing nanoparticles. Following release, the sample containing the streptavidin containing nanoparticles is added to the biotinylated biosensor followed by quantification of the number of streptavidin containing nanoparticles that bind the biotinylated biosensor using an imaging platform.

In a second exemplary embodiment of the assay, the nanoparticles of the method are tethered to the surface of a biosensor using nucleotide tethers. Assay medium is then added to the assay. The assay medium, comprising a guide polynucleotide sequence and a Cas enzyme, wherein the guide polynucleotide sequence and the Cas enzyme are capable of forming a CRISPR/Cas complex when exposed to a sample containing a target nucleotide sequence or also added to the assay. In addition, one of skill in the art will understand that the assay medium may also contain components required for harvesting, storing, or preserving the collected samples and/or biological samples. The sample and/or biological sample can be any sample suspected of containing a nucleotide sequence. One of skill in the art will understand that this includes, but is not limited, to tissue and/or body fluids from any mammal. In a preferred embodiment the samples are from a human. In some embodiment the sample is from a non-mammal host, which may contain the target nucleotide sequence. Upon addition of the sample that may contain the target nucleotide sequence to the assay, a CRISPR/Cas complex is formed followed by quantification of the number of nanoparticles tethered to the biosensor before and after addition of the sample using an imaging platform. In a preferred embodiment the imaging system is a PRAM imaging platform. The imaging platform can further comprise alternative non-imaging detection instruments. The imaging platform can also be a fluorescent microscope, TIRF microscope, dark field microscope, electron microscope, atomic force microscope, or reflection interference microscope.

Further, the second embodiment provided herein has the additional surprising technical effect of providing a result in less than twenty minutes when using the PRAM imaging system as part of the system, assay, or method disclosed herein allowing for rapid detection of the presence of viral infection or disease, when the concentration of the target molecule is sufficiently high. As such the systems, assays, and methods described herein can be used at the point of care.

### EXAMPLES

The Examples that follow are illustrative of specific embodiments of the invention, and various uses thereof. They are set forth for explanatory purposes only and should not be construed as limiting the scope of the disclosure in any way.

Methods

### PC Surface Silanization

Surface functionalization was achieved using oxygen plasma to chemically activate the exterior titanium oxide layer of the photonic crystal (PC). Reactive hydroxyl groups generated by plasma treatment were subsequently derivatized by liquid-phase silanization at room temperature for 30-minutes using a silane mixture suspended in anhydrous tetrahydrofuran (THF). The 50 mL solution was comprised of 49 mL THF, 900 uL of 3-(triethoxysilyl)propyl isocyante, 50 uL of butyl(chloro)dimethylsilane, and 50 uL of chloro(dimethyl)octylsilane. Following silanization, PC surfaces used to capture cleaved AuNPs underwent secondary functionalization for 12 hours at room temperature by reaction with amine—PEG₁₁—biotin at a concentration of 10 mg/mL in phosphate buffered saline containing 0.5% N,N-diisopropylethylamine (DIPEA).

### AuNP Surface Preparation

Amine/biotin-capped ssDNA tethers were incubated with streptavidin-conjugated gold nanoparticles (AuNPs) at 30° C. for 30 minutes in 1 mM hydroquinone (suspended in nuclease-free water) and isolated after centrifugation at 1,200 rcf for 10 minutes. The ssDNA-conjugated AuNPs were then resuspended in 1 mM hydroquinone and sonicated for 30 seconds.

### AuNP Immobilization on PC Surface

ssDNA-conjugated AuNPs suspended in 150 uL of 1 mM hydroquinone buffer were immobilized on silanized PC surfaces by co-incubation at room temperature for 30 minutes. After immobilization, PC surfaces were washed sequentially in four 50 mL aliquots of 1 mM hydroquinone.

### Assembly of Cas12a-sgRNA Complex

Equal volumes of 100 nM enzyme (EnGen® Lba Cas12a) and 125 nM sgRNA were mixed together in 1× CutSmart® buffer (diluted in nuclease-free water). The solution was allowed to incubate at 4° C. for 1 hour allowing for assembly of the Cas12a-sgRNA complex.

### Target Activation of Cas12a-sgRNA Complex

Assembled Cas12a-sgRNA complexes were activated by co-incubation with synthetic mutant dsDNA EGFR gene fragments in 150 uL of 1× CutSmart® buffer at 37° C. for two minutes. Each aliquot of activated complex was then added to a separate 1.5 mL Eppendorf centrifuge tube containing a 3×4 mm PC with immobilized AuNPs.

### Surface Cleavage and Capture of AuNPs

PCs immersed in target-activated Cas12a-sgRNA solution were incubated at 37° C. for one hour and then removed from the solution. The target-activated solution containing AuNPs released from cleavage was then transferred to a separate 1.5 mL Eppendorf tube containing an amine—PEG₁₁—biotin functionalized capture PC. The capture PC was incubated for one hour in the solution containing released AuNPs and washed before imaging.

### Surface Imaging and Particle Counting

After washing, PC surfaces used for AuNP capture were irradiated by a 617 nm laser and imaged under a 50× microscope objective. Particle counts were measured after post-processing of the acquired images.

### Example 1: Preliminary Validation of CRISPR Assay Components in a Fluorescence Test Demonstrates the Ability of RNP Complex to Cleave the Reporter Sequence

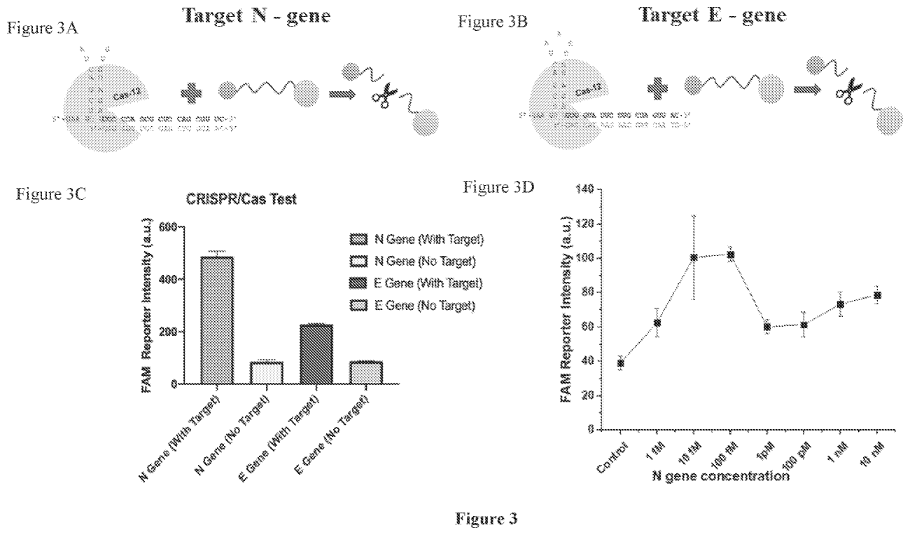

A preliminary validation of the CRISPR assay components were conducted in the presence of a reporter sequence, which consisted of 6-Carboxyfluorescein (6-FAM) on one end and Black Hole Quencher (BHQ) on the other end. The activated RNP complex for both sections of the SARS-CoV-2 genome (denoted as N and E genes in FIG. 3(A) and FIG. 3(B), respectively) demonstrated the ability to cleave the reporter sequence. Consequently, a significant increase in the fluorescence emission intensity of the FAM reporter was observed in FIG. 3(C). The same assay was repeated by varying the concentration of the target N-gene concentration. As shown in FIG. 3(D), no specific pattern in the fluorescence emission intensity was observed when the target gene concentration was varied between 1 fM to 10 nM.

### Example 2: CRISPR Assay Reveals Successful Cleavage of the DNA Tether in the Presence of the Activated RNP Complex

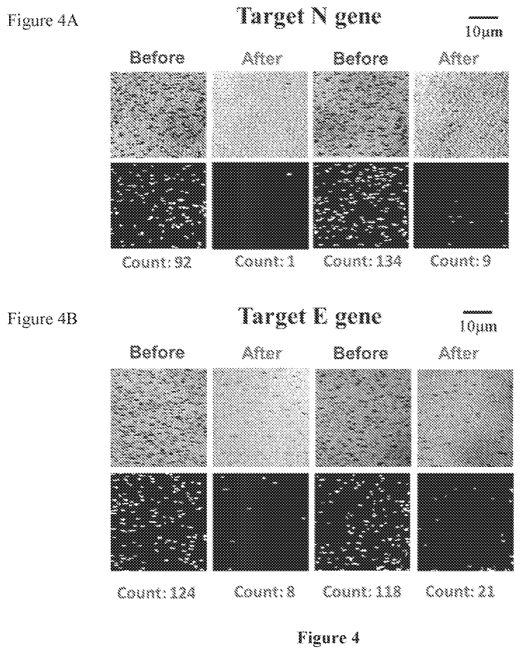

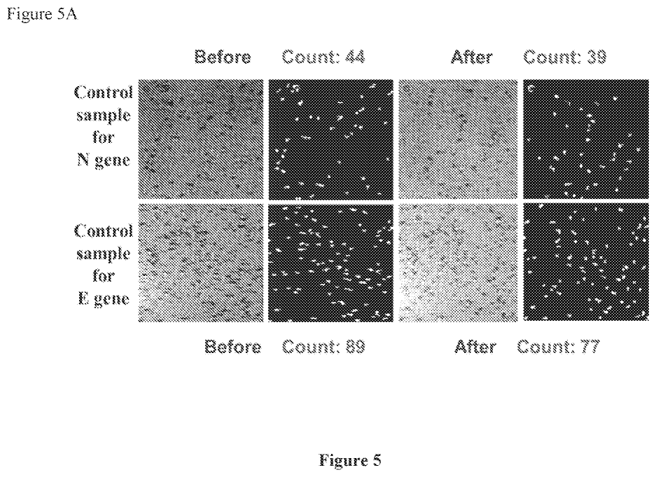

Following the preliminary validation of the CRISPR components, the assay was conducted on the PC using the principle illustrated in FIG. 2. The detection assay consisted of the following components: (i) PC functionalized with AuNPs attached via DNA tethers (ii) Activated RNP complex: Cas 12a+guide RNA for defined target+target gene (iii) Molecular grade water for the purpose of washing detached AuNPs. The activated RNP complex was added to a polydimethylsiloxane (PDMS) well, adhering to the PC, and incubated for 10 minutes. Subsequently, the well was then washed three times with molecular grade water. AuNP images before the addition of the activated RNP complex and after washing with water were captured using the portable PRAM. For both sections of the SARS-CoV-2 genome, a decrease in AuNP counts were observed after the addition of the activated RNP complex. This phenomenon was attributed to the successful cleavage of the DNA tether connecting the AuNPs on the PC surface in the presence of the activated RNP complex. Changes in AuNP count results for target N gene and target E gene have been shown in FIGS. 4(A) and 4(B) respectively.

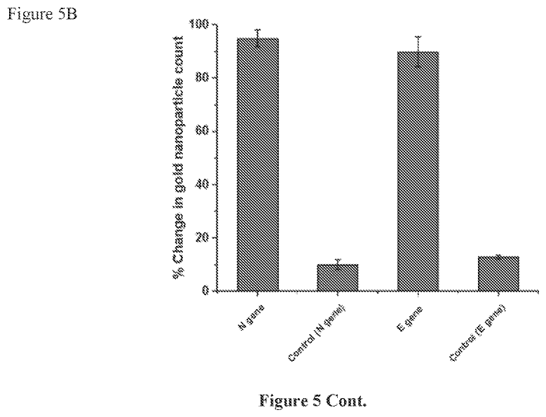

The specificity of the assay was tested by comparing the cleavage activity of activated RNP complex with that of the non-activated complex. While the activated complex consisted of the target gene, the non-activated complex (denoted as control in FIG. 5 (A, B)) did not have the target gene in it. As observed in FIG. 5(A), for both sections of the SARS-CoV-2 genome, there was no significant change in the AuNP counts before and after the addition of the inactivated CRISPR complex on the PC. The chart shown in FIG. 5(B) shows that approximately 95% of AuNPs were removed in the presence of the activated RNP complex while a change of approximately 12% of AuNPs was observed when the target gene was absent in the control sample.

### Example 3: Capture of Nanoparticles Released by Cas12a Tether Cleavage

The capture-based assay began with target activation of the RNP complex and subsequent cleavage of ssDNA tethered AuNPs found on individual 3×4 mm PCs inside separate 1.5 mL tubes containing using 150 μL of RNP complex with a known concentration of target gene at 37° C. for one hour. Following incubation, the 150 μL solution of RNP complex, gene fragments and released AuNPs was transferred into an 8×8 mm circular well with 200 μL volume capacity containing a capture PC surface functionalized with biotinylated PEG. The AuNPs suspended in the 150 uL solution were incubated in the well containing the capture PC for one hour, which were then removed from the solution and imaged on the portable PRAM using a 50× objective. Negative control PCs incubated with RNP complex containing no target gene fragments were imaged to determine AuNP counts associated with non-specific cleavage (background signal). Each capture PC incubated with samples containing RNP complex and gene fragments, either EGFR^{WT} (control) or EGFR^{L858R} (mutant), were imaged to obtain AuNP counts. After subtracting average background signal measured by imaging negative control PCs, absolute AuNP counts were obtained for each PC, respective of concentration (mol/L) and sequence identity (EGFR^{WT} or EGFR^{L858R}). Dose response curves were then constructed for each gene fragment (control and mutant) using their respective AuNP counts. No dose response was observed for control samples, while in the case of mutant gene fragments, a linear dose response was achieved over a concentration range of 1 zM-1 fM (FIGS. 7-9).

### CONCLUSION

The current disclosure demonstrates the successful and rapid detection of various sections of the SARS-CoV-2 genome on the PRAM PC based biosensing platform. The flexible nature of the CRISPR assay components, highlights the use of the system, assay, and methods described herein for the detection of infectious disease and agents as well as pathological conditions that impact human health such as human cancer.

### REFERENCES

1. C. Zhang and D. Xing, “Miniaturized PCR chips for nucleic acid amplification and analysis: latest advances and future trends,” Nucleic Acids Res., vol. 35, no. 13, pp. 4223-4237, 2007.
2. J. Wang, Z. Chen, P. L. A. M. Corstjens, M. G. Mauk, and H. H. Bau, “A disposable microfluidic cassette for DNA amplification and detection,” Lab Chip, vol. 6, no. 1, pp. 46-53, 2006.
3. S. H. Lee, S.-W. Kim, J. Y. Kang, and C. H. Ahn, “A polymer lab-on-a-chip for reverse transcription (RT)-PCR based point-of-care clinical diagnostics,” Lab Chip, vol. 8, no. 12, pp. 2121-2127, 2008.
4. “Types of Zika Virus Tests.,” Center for Disease Control (CDC). [Online]. Available: http://www.cdc.gov/zika/laboratories/types-of-tests.html.
5. R. N. Charrel, I. Leparc-Goffart, S. Pas, X. de Lamballerie, M. Koopmans, and C. Reusken, “Background review for diagnostic test development for Zika virus infection,” Bull. World Health Organ., vol. 94, no. 8, p. 574, 2016.
6. N. Bonne, P. Clark, P. Shearer, and S. Raidal, “Elimination of false-positive polymerase chain reaction results resulting from hole punch carryover contamination,” J. Vet. Diagnostic Investig., vol. 20, no. 1, pp. 60-63, 2008.
7. G. J. Knott et al., “Guide-bound structures of an RNA-targeting A-cleaving CRISPR-Cas13a enzyme,” Nat. Struct. Mol. Biol., vol. 24, no. 10, p. 825, 2017.
8. J. S. Chen et al., “CRISPR-Cas12a target binding unleashes indiscriminate single-stranded DNase activity,” Science (80-.)., vol. 360, no. 6387, pp. 436-439, 2018.
9. T. D. Canady et al., “Digital-resolution detection of microRNA with single-base selectivity by photonic resonator absorption microscopy,” Proc. Natl. Acad. Sci., vol. 116, no. 39, pp. 19362-19367, 2019.

What is claimed is:

1. A system for detecting nucleic acids in a sample, comprising:
a source substrate with streptavidin linked nanoparticles bound to the surface of the source substrate by nucleotide tethers;
an assay medium comprising a guide polynucleotide sequence and a Cas enzyme, wherein the guide polynucleotide sequence and the Cas enzyme are capable of forming a CRISPR/Cas complex when exposed to a sample containing a target nucleotide sequence;
a biotinylated biosensor;
an imaging platform;
wherein the guide polynucleotide sequence binds the target nucleotide sequence and Cas enzyme thereby forming the CRISPR/Cas complex;
wherein the Cas enzyme is configured to cleave the nucleotide tethers thereby releasing the streptavidin linked nanoparticles;
wherein the streptavidin linked nanoparticles bind the biotinylated biosensor, and
wherein the imaging platform is configured to quantify the number of streptavidin linked nanoparticles bound to the biotinylated biosensor.

2. The system of claim 1, wherein the substrate source is a biologically inert solid material.

3. The system of claim 2, wherein the inert solid material is glass (silicon oxide).

4. The system of claim 2, wherein the inert solid material is a plastic comprised of one or more of polyester, polystyrene, or acrylic.

5. The system of claim 2, wherein the inert solid material is gold or silver.

6. The system of claim 2, wherein the inert solid material is silicon nitride or titanium oxide.

7. The system of claim 1, wherein the Cas is Cas 9, Cas12a, Cas12b, or Cas13.

8. The system of claim 1, wherein the target nucleotide sequence is indicative of SARS-CoV2.

9. The system of claim 1, wherein the target nucleotide sequence is indicative of cancer.

10. The system of claim 9, wherein the cancer is a solid tumor

11. The system of claim 10, wherein the cancer is a blood-based cancer.

12. The system of claim 1, wherein the biosensor comprises a photonic crystal, and wherein the imaging platform comprises a light source configured to excite a resonance of the photonic crystal and a detector configured to detect light reflected from the photonic crystal.

13. The system of claim 12, wherein the imaging platform is configured to quantify a resonant peak intensity value measured on a pixel-by-pixel basis across the photonic crystal.

14. The system of claim 1, wherein the imaging platform comprises a non-imaging detection instrument.

15. The system of claim 1, wherein the biosensor is a whispering gallery mode biosensor.

16. The system of claim 15, wherein the whispering gallery biosensor is a ring resonator, microtoroid, or microsphere.

17. The system of claim 1, wherein the biosensor comprises a waveguide structure through which light travels laterally.

18. The system of claim 1, wherein the biosensor is an acoustic biosensor.

19. The system of claim 1, wherein the biosensor is a photoacoustic biosensor.

20. The system of claim 1, wherein the biosensor is a surface plasmon resonant biosensor.

21. A biologic assay comprising:
a source substrate;
a biotinylated biosensor;
assay medium comprising a guide polynucleotide sequence and a Cas enzyme,
a population of streptavidin linked nanoparticles; and
a plurality of nucleotide tethers;
wherein the streptavidin-linked nanoparticles are bound to the biosensor using the plurality of nucleotide tethers, and wherein the nucleotide tethers are comprised of a nucleic acid sequence.

22. The biologic assay of claim 21 wherein a sample containing a nucleotide target sequence for detection complementary to the guide polynucleotide sequence is added to the assay and is capable of forming an activated CRISPR/Cas complex.

23. The biological assay of claim 21, wherein the Cas enzyme is Cas9 and the nucleotide target sequence for detection is a messenger RNA (mRNA).

24. The biological assay of claim 21, wherein the Cas enzyme is Cas 13 and the nucleotide target sequence for detection is a microRNA (miRNA).

25. The biological assay of claim 21 wherein the Cas enzyme is Cas 12 and the nucleotide target sequence for detection is a double stranded DNA (dsDNA).

26. The biologic assay of claim 21, wherein the nanoparticles are gold nanoparticles, quantum dots, metal-based nanoparticles, magnetic nanoparticles, magnetic-plasmonic nanoparticles, phosphorescent nanoparticles, or nanoparticles comprised of dielectric materials such as SiO₂ or TiO₂.

27. The biologic assay of claim 21, wherein the biosensor comprises a photonic crystal, further comprising an imaging platform configured to quantify a resonant peak intensity value measured on a pixel-by-pixel basis across the photonic crystal.

28. The biologic assay of claim 21, wherein the biosensor comprises a non-imaging detection instrument.

29. The biologic assay of claim 21, wherein the biosensor is a whispering gallery mode biosensor.

30. The biologic assay of claim 29, wherein the whispering gallery biosensor is a ring resonator, microtoroid, or microsphere.

31. The biologic assay of claim 21, wherein the biosensor comprises a waveguide structure through which light travels laterally.

32. The biologic assay of claim 21, wherein the biosensor is an acoustic biosensor.

33. The biologic assay of claim 21, wherein the biosensor is a photoacoustic biosensor.

34. The biologic assay of claim 21, wherein the biosensor is a surface plasmon resonant biosensor.

35. The biologic assay of claim 21, wherein the nucleotide tethers are comprised of nucleotide sequences between 4 and 100 nucleotides in length.

36. The biologic assay of claim 35, wherein the nucleotide tethers are homogenous in nucleotide length.

37. The biologic assay of claim 35, wherein the nucleotide tethers are heterogenous in nucleotide length.

38. The biologic assay of claim 21, wherein the CAS enzyme is Cas 9, Cas12a or Cas13a.

39. The biologic assay of claim 21, wherein the biologic assay is stable at room temperature.

40. A method for detecting nucleic acids in a sample, comprising the steps of:
binding streptavidin to a nanoparticle to create a streptavidin containing nanoparticle;
tethering the streptavidin containing nanoparticles to the surface of a source substrate using nucleotide tethers, thereby creating an assay surface;
coating a biosensor with biotin, thereby creating a biotinylated biosensor;
generating an activated Cas enzyme by adding a test sample to an assay medium,
wherein the assay medium comprises a guide polynucleotide sequence and a Cas enzyme,
wherein the guide polynucleotide sequence and the Cas enzyme are capable of forming an activated CRISPR/Cas complex when exposed to the test sample containing a target nucleotide sequence;
capturing streptavidin containing nanoparticles cleaved upon incubation of the activated Cas enzyme and assay surface;
incubating the cleaved streptavidin containing nanoparticles with the biotinylated biosensor; and
quantifying the number of streptavidin containing nanoparticles that bind the biotinylated biosensor using an imaging platform.

41. The method of claim 40, wherein the Cas enzyme is Cas 9, Cas12 or Cas13.

42. The method of claim 40, wherein the quantity of streptavidin containing nanoparticles bound to the biotinylated biosensor is indicative of the presence of a target RNA or DNA molecule, whose sequence is a biomarker for disease, the presence of a viral pathogen, or the presence of a bacterial pathogen.

43. The method of claim 40, wherein the target nucleotide sequence is indicative of SARS-CoV2.

44. The method of claim 40, wherein the target nucleotide sequence is indicative of cancer.

45. The method of claim 44, wherein the cancer is a solid tumor.

46. The method of claim 44, wherein the cancer is a blood-based cancer.

47. The method of claim 40, wherein the biosensor comprises a photonic crystal, and wherein the imaging platform comprises a light source configured to excite a resonance of the photonic crystal and a detector configured to detect light reflected from the photonic crystal.

48. The method of claim 47, wherein the imaging platform is configured to quantify a resonant peak intensity value measured on a pixel-by-pixel basis across the photonic crystal.

49. The method of claim 40, wherein the imaging platform comprises a non-imaging detection instrument.

50. The method of claim 40, wherein the imaging platform is a fluorescent microscope, TTRF microscope, dark field microscope, electron microscope, atomic force microscope, reflection interference microscope.

51. The method of claim 40, wherein the biosensor is a whispering gallery mode biosensor.

52. The method of claim 51, wherein the whispering gallery biosensor is a ring resonator, microtoroid, or microsphere.

53. The method of claim 40, wherein the biosensor comprises a waveguide structure through which light travels laterally.

54. The method of claim 40, wherein the biosensor is an acoustic biosensor.

55. The method of claim 40, wherein the biosensor is a photoacoustic biosensor.

56. The method of claim 40, wherein the nanoparticles are gold nanoparticles, quantum dots, metal-based nanoparticles, magnetic nanoparticles, magnetic-plasmonic nanoparticle tags or nanoparticles comprised of dielectric materials such as SiO₂ or TiO₂.

57. A system for detecting nucleic acids in a sample, comprising:
streptavidin linked nanoparticles bound to free floating microparticles by nucleotide tethers;
an assay medium comprising a guide polynucleotide sequence and a Cas enzyme, wherein the guide polynucleotide sequence and the Cas enzyme are capable of forming a CRISPR/Cas complex when exposed to a sample containing a target nucleotide sequence;
a biotinylated biosensor;
an imaging platform;
wherein the guide polynucleotide sequence binds the target nucleotide sequence and Cas enzyme thereby forming the CRISPR/Cas complex;
wherein the Cas enzyme is configured to cleave the nucleotide tethers thereby releasing the streptavidin linked nanoparticles;
wherein the streptavidin linked nanoparticles bind the biotinylated biosensor, and
wherein the imaging platform is configured to quantify the number of streptavidin linked nanoparticles bound to the biotinylated biosensor.

58. A biologic assay comprising:
streptavidin linked nanoparticles;
free floating microparticles;
a biotinylated biosensor;
assay medium comprising a guide polynucleotide sequence and a Cas enzyme,
a population of streptavidin linked nanoparticles; and
a plurality of nucleotide tethers;
wherein the streptavidin-linked nanoparticles are bound to the free floating microparticles using the plurality of nucleotide tethers, and wherein the nucleotide tethers are comprised of a nucleic acid sequence.

59. A method for detecting nucleic acids in a sample, comprising the steps of:
binding streptavidin to a nanoparticle to create a streptavidin containing nanoparticle;
tethering the streptavidin containing nanoparticles to free floating microparticles using nucleotide tethers,
coating a biosensor with biotin, thereby creating a biotinylated biosensor;
generating an activated Cas enzyme by adding a test sample to an assay medium,
wherein the assay medium comprises a guide polynucleotide sequence and a Cas enzyme,
wherein the guide polynucleotide sequence and the Cas enzyme are capable of forming an activated CRISPR/Cas complex when exposed to the test sample containing a target nucleotide sequence;
capturing streptavidin containing nanoparticles cleaved upon incubation of the activated Cas enzyme and free floating microparticles;
incubating the cleaved streptavidin containing nanoparticles with the biotinylated biosensor; and
quantifying the number of streptavidin containing nanoparticles that bind the biotinylated biosensor using an imaging platform.

60. A system for detecting nucleic acids in a sample, comprising:
a biosensor with nanoparticles bound to the surface of the biosensor by nucleotide tethers;
an assay medium comprising a guide polynucleotide sequence and a Cas enzyme, wherein the guide polynucleotide sequence and the Cas enzyme are capable of forming a CRISPR/Cas complex when exposed to a sample containing a target nucleotide sequence; and
an imaging platform,
wherein the guide polynucleotide sequence binds the target nucleotide sequence and Cas enzyme thereby forming the CRISPR/Cas complex;
wherein the Cas enzyme is configured to cleave the nucleotide tethers thereby releasing nanoparticles; and
wherein the imaging platform is configured to quantify the number of nanoparticles tethered to the biosensor prior to and after addition of the sample.

61. A biologic assay comprising:
a biosensor;
assay medium comprising a guide polynucleotide sequence and a Cas enzyme,
a population of nanoparticles; and
a plurality of nucleotide tethers;
wherein the nanoparticles are bound to the surface of the biosensor using the plurality of nucleotide tethers, and wherein the nucleotide tethers are comprised of a nucleic acid sequence.

62. A method for detecting nucleic acids in a sample, comprising the steps of:
tethering nanoparticles to the surface of a biosensor using nucleotide tethers, thereby creating an assay surface;
adding an assay medium to the assay surface, wherein the assay medium comprises a guide polynucleotide sequence and a Cas enzyme, wherein the guide polynucleotide sequence and the Cas enzyme are capable of forming a CRISPR/Cas complex when exposed to a sample containing a target nucleotide sequence;
adding a biological sample that may contain the target nucleotide sequence to the assay, thereby forming a CRISPR/Cas complex; and
quantifying the number of nanoparticles tethered to the biosensor before and after addition of the sample using an imaging platform.

63. The method of claim 62, wherein after quantification of the tethered nanoparticles the nanoparticles are removed from the biosensor surface.

64. The method of claim 62, wherein the nanoparticles are removed from the biosensor surface by replacing the assay buffer.

65. The method of claim 62, wherein the nanoparticle is removed from the biosensor surface by agitation of the assay buffer without replacement of the assay buffer.

66. The method of claim 62 wherein if the nanoparticle is a magnetic nanoparticle, the nanoparticle is removed from the biosensor surface by application of a magnetic field.
